(19) United States
(12) Patent Application Publication
(10) Pub. No.: US 2024/0368324 A1
(43) Pub. Date: Nov. 7, 2024

(54) PLANT-INSPIRED ZWITTERIONIC MONOMERS, POLYMERS, AND USES THEREOF

(71) Applicant: Trustees of Tufts College, Medford, MA (US)

(72) Inventors: Matthew Panzer, Medford, MA (US); MOrgan Taylor, Medford, MA (US); Ayse Asatekin Alexiou, Arlington, MA (US)

(21) Appl. No.: 18/572,390

(22) PCT Filed: Jun. 24, 2022
(86) PCT No.: PCT/US2022/034908

§ 371 Date: Dec. 20, 2023

Related U.S. Application Data
(60) Provisional application No. 63/215,781, filed on Jun. 28, 2021.

(51) Int. Cl. C08F 226/06; A61L 26/00; A61L 27/34; A61L 27/52; B01D 71/76; C08F 220/36; H01M 10/0565

(52) U.S. Cl. CPC C08F 226/06; A61L 26/0014; A61L 26/008; A61L 27/34; A61L 27/52; B01D 71/76; C08F 220/365; H01M 10/0565

(57) ABSTRACT

Disclosed are polymers comprising a plurality of monomers, wherein at least some of the monomers are zwitterions that comprise a betaine having a pyridinium group and a carboxylate group. Also disclosed are filtration membranes, coating materials, wound dressings, electrolytes, batteries, and formulations comprising such polymers. Additionally disclosed are methods of preparing such polymers.

### RELATED APPLICATION

This application claims the benefit of priority to U.S. Provisional Application No. 63/215,781, filed Jun. 28, 2021; the contents of which is incorporated by reference.

### GOVERNMENT SUPPORT

This invention was made with government support under Grant Number 1802729, awarded by the National Science Foundation. The government has certain rights in the invention.

### BACKGROUND

Zwitterionic (ZI) polymers are a diverse subclass of materials that are the focus of research for numerous fields including: drug delivery, bio-implants, anti-fouling materials, and electrochemical energy storage. There have been multiple distinct types of zwitterion chemistries and materials that highlight their unique properties and potential for battery electrolytes.

However, commercially available ZI monomers include a very limited selection of functional groups, such as sulfobetaine-type (e.g. sulfobetaine methacrylate, SBMA) that do not enhance Li⁺ transport, and phosphorylcholine-type (e.g., 2-methacryloyloxyethyl phosphorylcholine, MPC) that are expensive to produce.

Therefore, a major disadvantage to widespread use of zwitterions is the limited number of chemistries that are commercially available or easy to synthesize. For this reason, there is a need to continue to develop new zwitterion chemistries, particularly containing carboxybetaine (CB) and phosphorylcholine (PC) motifs that also lower the synthetic barrier and increase zwitterion availability for future applications.

### SUMMARY

In some aspects, the present invention provides a polymer, comprising a plurality of monomers, wherein at least some of the monomers are zwitterions that comprise a betaine having a pyridinium group and a carboxylate group.

In certain embodiments, the polymer is a hydrogel.

In certain embodiments, the carboxylate group is linked to C3 of said pyridinium group.

In certain embodiments, the zwitterions further comprise an alkyl, allyl, aryl, vinylbenzyl, acrylate, methacrylate, acrylamide, or a methacrylamide group.

In certain embodiments, the zwitterions comprise:

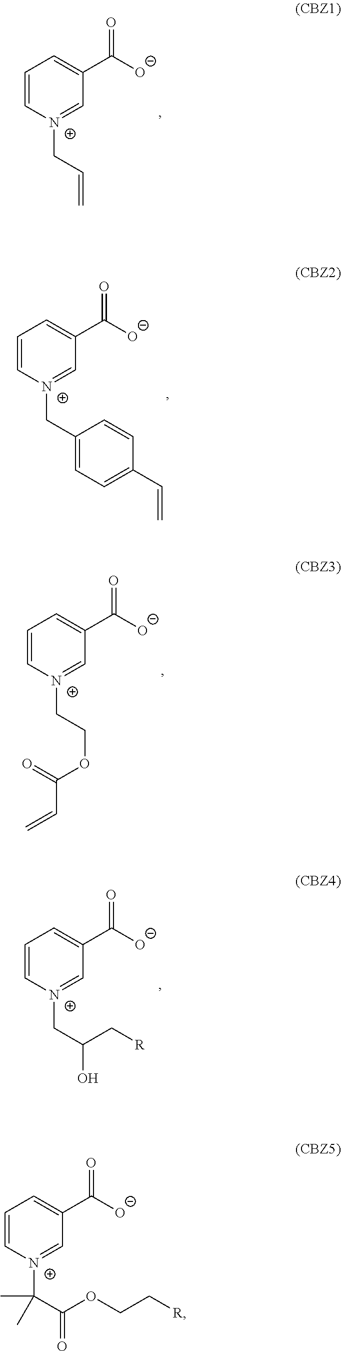

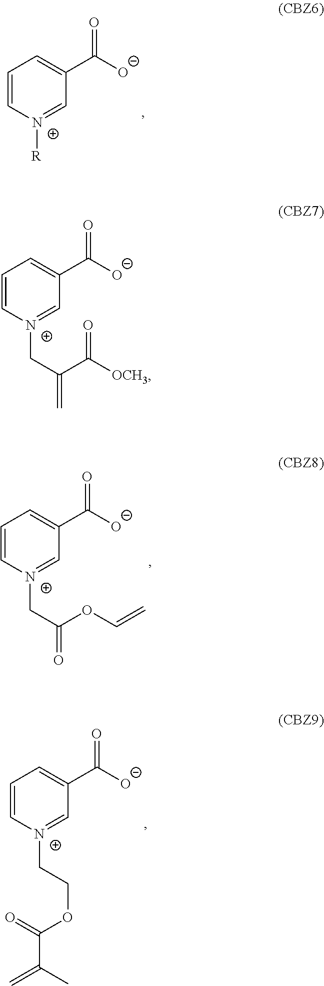

or a combination of any of them; and
R represents acrylate (—OC(O)CH═CH₂), methacrylate (—OC(O)C(CH₃)═CH₂), acrylamide (—NHC(O)CH═CH₂), or methacrylamide (—NHC(O)C(CH₃)═CH₂).

In certain embodiments, the polymer is a copolymer further comprising hydrophobic monomers, charged monomers, ionizable monomers, or a combination of any of them.

In other aspects, the invention provides a filtration membrane (e.g., a water filtration membrane) comprising the polymer of the invention.

In other aspects, the invention provides a coating material (e.g., a bio-implant coating material, an implant surface coating material, a biomedical device coating material, an anti-fouling material) comprising the polymer of the invention.

In other aspects, the invention provides a wound-dressing material comprising the polymer of the invention.

In other aspects, the invention provides an ionic liquid-based electrolyte (e.g., ionogel electrolyte) or a polymer electrolyte comprising the polymer of the invention.

In other aspects, the invention provides Li-ion batteries comprising the ionic liquid-based electrolyte or polymer electrolyte of the invention.

In other aspects, the invention provides drug delivery formulations comprising the polymer of the invention.

In some aspects, the invention provides methods of preparing a carboxybetaine monomer comprising reacting nicotinic acid with an electrophile to obtain a cationic intermediate; and reacting the cationic intermediate with a base to obtain the carboxybetaine monomer.

In certain embodiments, the method further comprises a solvent, e.g., DMF.

In certain embodiments, the electrophile is a halide or an epoxide.

In certain embodiments, the electrophile is

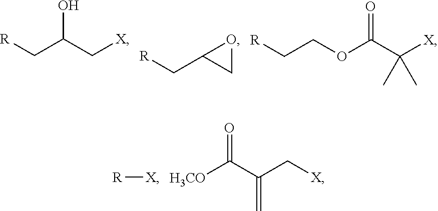

or

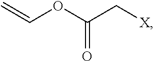

and R is substituted or unsubstituted alkyl, allyl, or vinyl, and X is a halogen (e.g., bromine, chlorine, fluorine, or iodine).

In certain embodiments, the electrophile is a halide; and the halide is allyl bromide, 4-vinylbenzyl chloride, or 2-chloroethyl acrylate.

In certain embodiments, the carboxybetaine monomer is:

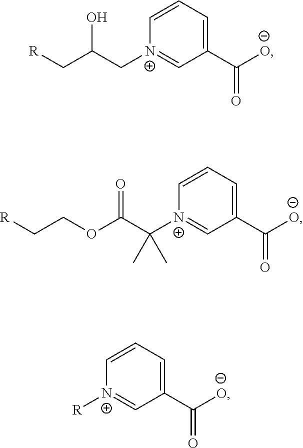

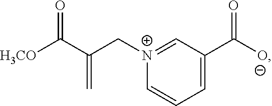

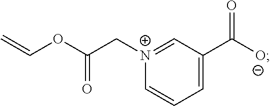

and R is substituted or unsubstituted alkyl, allyl, or vinyl.

In certain embodiments, the cationic intermediate is:

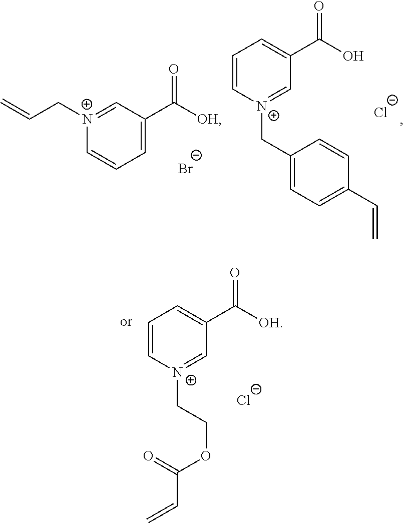

In certain embodiments, the base is an alkali hydroxide (e.g., sodium hydroxide).

In certain embodiments, the carboxybetaine monomer is:

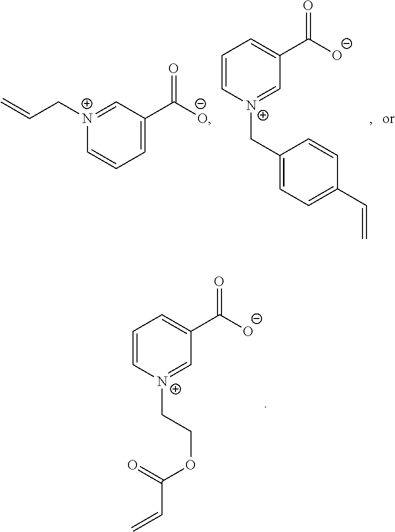

In some aspects, methods of preparing a polymer comprising carboxybetaine monomers comprise polymerizing a plurality of carboxybetaine monomers obtained by reacting nicotinic acid with a halide to obtain a cationic intermediate; and reacting the cationic intermediate with a base to obtain the carboxybetaine monomer.

These and other aspects of the present disclosure will become apparent upon a review of the following detailed description and the claims.

### BRIEF DESCRIPTION OF THE FIGURES

FIG. 1: Synthesis schemes of bio-inspired zwitterion monomers. (a) CBZ1 prepared by reacting nicotinic acid with allyl bromide and (b) CBZ2 prepared by reacting nicotinic acid with 4-vinylbenzyl chloride.

FIG. 2: Photographs of monomer solutions containing new CB-type zwitterions. CBZ1 (left) and CBZ2 (right) in 1 M LiTFSI/BMP TFSI with a 0.3 ZI unit:Li⁺ mole ratio. At this mole ratio, the approximate concentrations were 22 mg CBZ1 and 33 mg CBZ2 in 500 μL 1M LiTFSI/BMP TFSI.

FIG. 3: ⁷Li NMR spectra of 1 M LiTFSI/BMP TFSI and zwitterion monomer solutions. Plot includes NMR spectra of 1 M LiTFSI/BMP TFSI solution (bottom) and ZI monomer solutions containing CBMA, CBZ1, SB2VP, and CBZ2

FIG. 4: Proposed synthesis scheme for CBZ3 by reacting nicotinic acid with 2-chloroethyl acrylate.

FIG. 5: ¹H NMR spectrum of the CBZ1 monomer. Synthesized by reacting nicotinic acid with allyl bromide following the procedure outlined in the experimental methods. Peak assignments are shown in the insert and NMR was performed using D₂O as the solvent. ¹H NMR (D₂O, 500 MHz): 9.14, 8.82, 8.79, 8.01 (m, Pr), 5.9-6.01 (1H, ═CH), 5.35-5.4 (2H, ═CH₂), 5.14 (2H, —CH₂N).

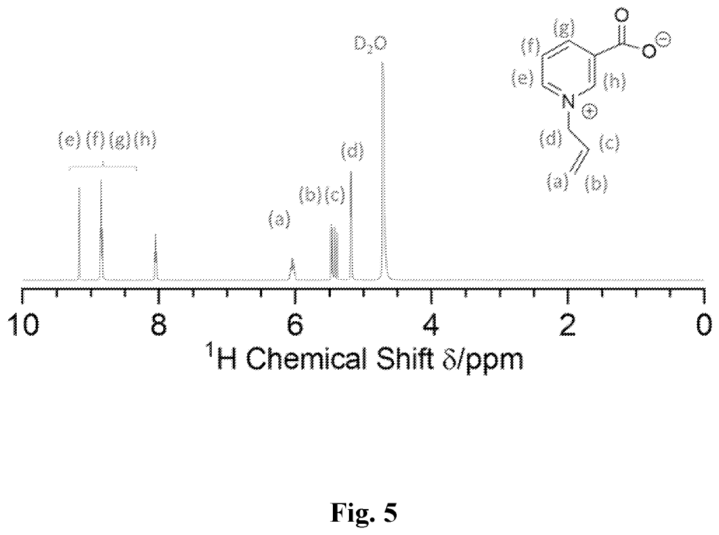

FIG. 6: ¹H NMR spectrum of the CBZ2 monomer. Prepared by reacting nicotinic acid with 4-vinylbenzyl chloride. Peak assignments are shown in the insert and NMR was performed using D₂O as the solvent. ¹H NMR (D₂O, 500 MHz): 8.75, 8.41, 8.05, 7.89 (m, Pr), 7.18-7.33 (m, benzene), 6.49-6.55 (2H, ═CH₂), 5.6 (1H, ═CH), 5.16 (2H, —CH₂N).

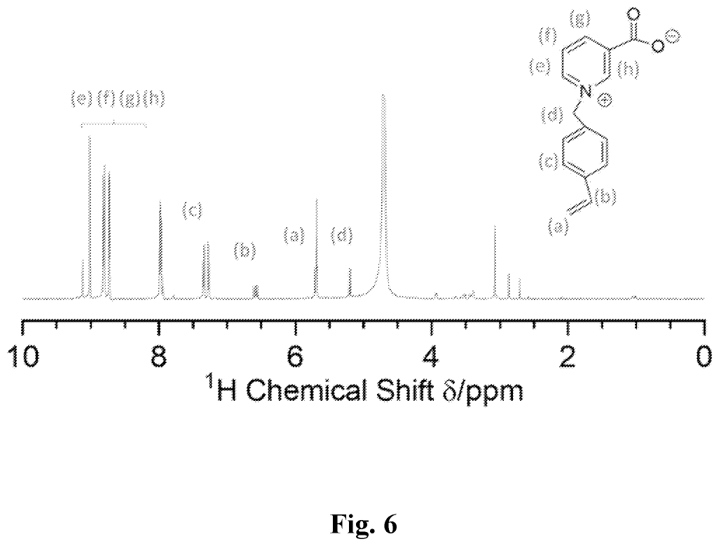

FIG. 7: ¹⁹F NMR spectra of 1 M LiTFSI/BMP TFSI solution and zwitterion monomer solutions. Figure includes NMR spectra of 1 M LiTFSI/BMP TFSI solution (bottom) and ZI monomer solutions containing CBMA, CBZ1, SB2VP and CBZ2. All samples contain specific ZI unit:Li⁺ mole fraction as indicated in the figure legend, and all samples are referenced to 0.5 M LiTFSI in D₂O at −79.15 ppm.

FIG. 8: Temperature dependence of ionic conductivity. Measured for 1 M LiTFSI/BMP TFSI solution (green) and electrolyte samples containing zwitterions CBZ1 (purple) and pCBZ2 (pink) with a 0.3 ZI unit:Li⁺ mole fraction. Calculated activation energy of ionic conductivity for each electrolyte is shown in the legend next to the name.

FIG. 9: Cell impedance responses (Nyquist plot) before and after polarization for the 1 M LiTFSI/BMP TFSI-based electrolytes. Samples include: (a) ionic liquid (IL) solution, (b) CBZ1 monomer solution, and (c) pCBZ2 gel. The concentration of zwitterion in (b) and (c) is a 0.3 ZI unit:Li⁺ mole fraction value, and insets show the chronoamperometry responses to an applied potential of 10 mV.

FIG. 10: Synthetic schemes for the reaction of niacin with various monomer building blocks to create zwitterionic monomers CBZ4, CBZ5, CBZ6, CBZ7, and CBZ8. R=acrylate (H₂C═CHC(O)O˜), methacrylate (H₂C═C(CH₃)C(O)O˜), acrylamide (H₂C═CHC(O)NH˜), or methacrylamide (H₂C═C(CH₃)C(O)NH˜) groups; X=Cl or Br. Arrows generally represent a two-step process (reaction to quaternize the nitrogen of niacin, followed by reaction with base to zwitterionize by deprotonating the carboxylic acid group).

FIG. 11: ¹H NMR spectrum of the CBZ9 monomer (acid version). Prepared by reacting nicotinic acid with 2-bromoethyl methacrylate. Peak assignments are shown in the inset and NMR was performed using D₂O as the solvent. ¹H NMR (D₂O, 500 MHz): 9.38, 9.00, 8.93, 8.11, (m, Pr), 5.60-5.96 (2H, ═CH₂), 4.96 (2H, —CH₂O), 4.60 (2H, —CH₂N), 1.73 (3H, —CH₃).

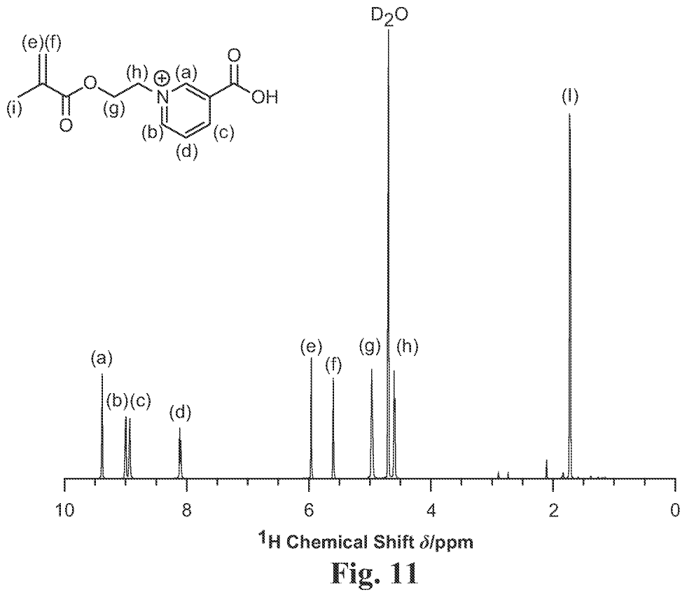

### DETAILED DESCRIPTION

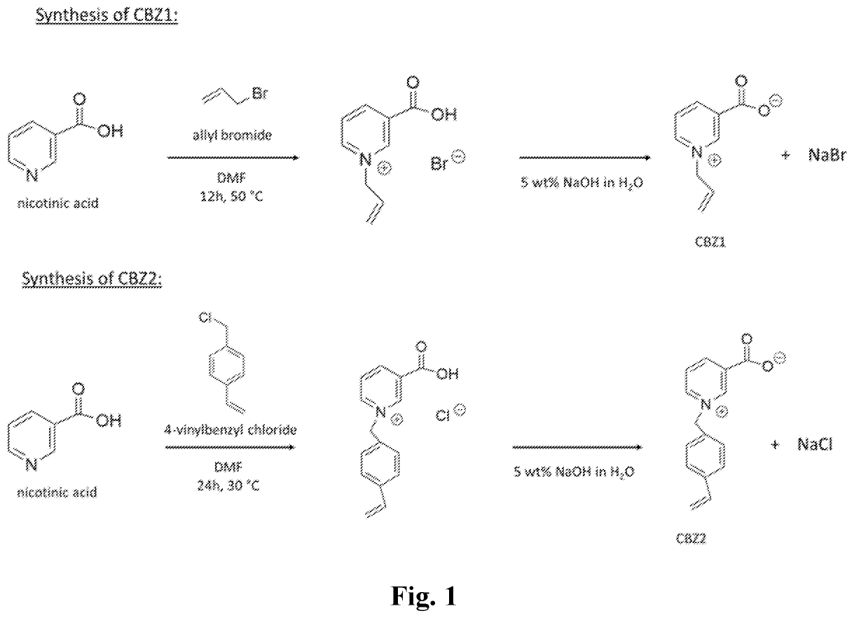

The present disclosure relates to CB-type ZI monomers have been synthesized, for the first time, in a simple two-step method (see, e.g., FIG. 1) from nicotinic acid as a precursor. An advantage of these materials is their simple synthesis using a naturally occurring reagent (nicotinic acid, niacin). Another potential advantage is the hydrophobicity of their pyridinium cationic unit, combined with the strongly Li⁺-coordinating carboxylate anionic unit.

The present disclosure describes a strategy for the chemical synthesis of a novel class of zwitterionic (ZI) monomers and their (co)polymers derived from a naturally-occurring and nontoxic, low-cost starting material: nicotinic acid, also known as niacin or one form of Vitamin B3. ZI monomers and their (co)polymers are practically important because of their anti-fouling properties, high degree of hydration, biocompatibility, and strong electrostatic interactions with ions. The disclosed experiments demonstrate the successful syntheses of different ZI monomers using nicotinic acid as a starting material, yielding novel ZI functional groups that were inspired by trigonelline (1-methylpyridin-1-ium-3-carboxylate), an alkaloid ZI small molecule found in several plants, including coffee plants (e.g., Coffea arabica). This specific ZI functional group has not been widely investigated or reported on with respect to synthetic monomers/(co)polymers to date. As such, it represents an important new addition to the ZI monomer/polymer community.

The carboxylate anionic unit of these ZI monomers has been shown to interact strongly with Li⁺ cations via NMR spectroscopy, and can improve Li⁺ conductivity in ionic liquid-based electrolytes (e.g. for Li-ion batteries). In addition, the relatively hydrophobic pyridinium cationic unit of these ZI monomers is expected to allow for enhanced tunability of nanopore properties in filtration membranes based on copolymer selective layers that incorporate these ZI units.

The ZI monomers and (co)polymers disclosed here represent a new class of carboxybetaine (CB)-type zwitterions. We have already demonstrated the ability of one such new homopolymer (pCBZ2) to improve Li⁺ conductivity inside an ionic liquid-based ionogel electrolyte (Table 1), comparable to another CB-type zwitterionic homopolymer that is more expensive and difficult to synthesize (pCBMA). Thus, this new class of monomers/(co)polymers can provide advantages for nonvolatile Li-ion battery gel electrolytes and possibly solid polymer electrolytes, as well. These materials will also allow to finely tune copolymer selective layers for water filtration applications, based on the combination of their CB type and hydrophobic pyridinium motif. More generally, these (co)polymers can be anti-fouling and biocompatible, leading to biomedical applications (such as wound dressings or implant surface coatings). The disclosures are also useful for battery development, water purification, and biomedical devices.

In an aspect, polymers comprise a plurality of monomers, wherein at least some of the monomers are zwitterions that comprise a betaine having a pyridinium group and a carboxylate group.

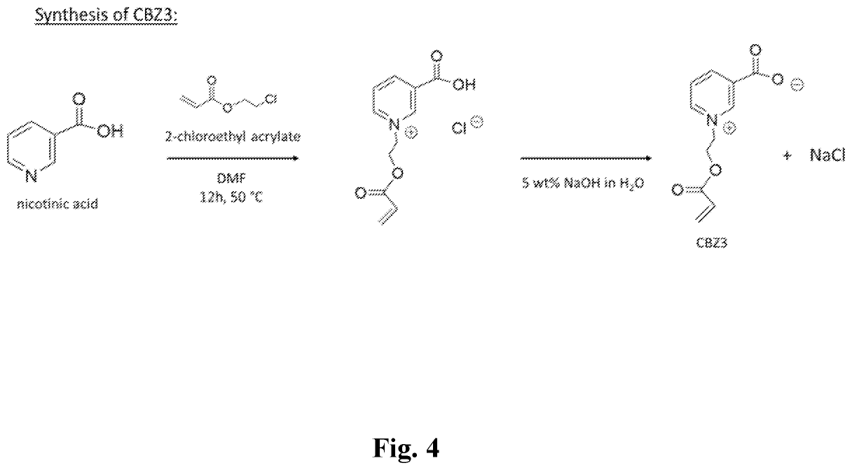

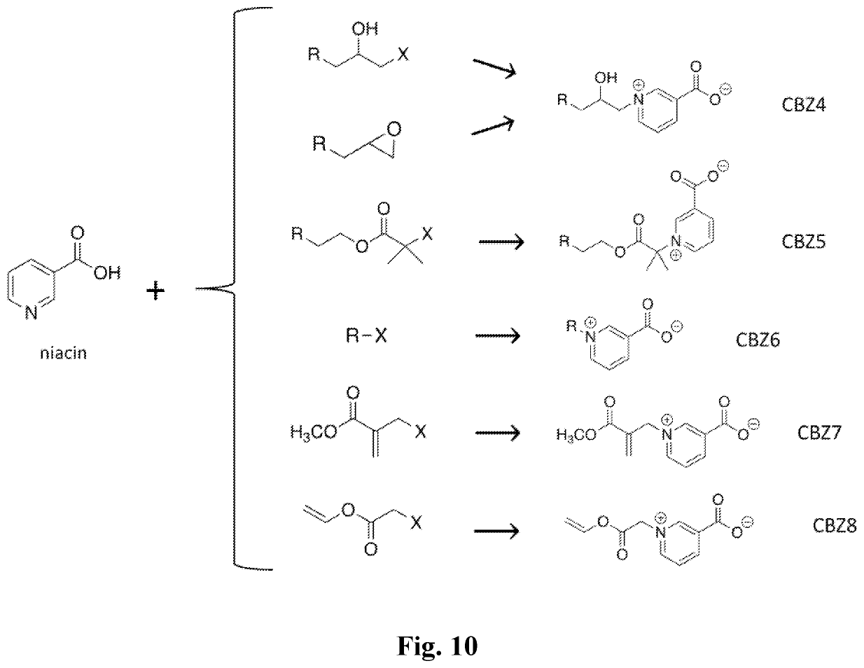

In some embodiments, the polymer is a hydrogel. In some embodiments, the carboxylate group is linked to C3 of the pyridinium group. In some embodiments, the zwitterions further comprise an alkyl, allyl, aryl, vinylbenzyl, acrylate, methacrylate, acrylamide, or a methacrylamide group. In some embodiments, the zwitterions comprise CBZ1 (as shown in FIG. 1), CBZ2 (as shown in FIG. 1), CBZ3 (as shown in FIG. 4), CBZ4 (as shown in FIG. 10), CBZ5 (as shown in FIG. 10), CBZ6 (as shown in FIG. 10), CBZ7 (as shown in FIG. 10), CBZ8 (as shown in FIG. 10), or a combination thereof. In some embodiments, the polymers are copolymers that further comprise hydrophobic monomers, charged monomers, ionizable monomers, or a combination thereof.

In some aspects, filtration membranes (e.g., water filtration membranes), coating materials (e.g., bio-implant coating materials, implant surface coating material, biomedical device coating materials, anti-fouling materials), wound-dressing materials, ionic liquid-based electrolytes (e.g., ionogel electrolytes), polymer electrolytes, Li-ion batteries with ionic liquid-based electrolytes or polymer electrolytes, or drug delivery formulations comprise the disclosed polymers.

In some aspects, methods of preparing a carboxybetaine monomer comprise reacting nicotinic acid with a halide to obtain a cationic intermediate; and reacting the cationic intermediate with a base to obtain the carboxybetaine monomer.

In some aspects, methods of preparing a polymer comprising carboxybetaine monomers comprise polymerizing a plurality of carboxybetaine monomers obtained by reacting nicotinic acid with a halide to obtain a cationic intermediate; and reacting the cationic intermediate with a base to obtain the carboxybetaine monomer.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

Articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

### Examples

The disclosure will be further illustrated with reference to the following specific examples. These examples are given by way of illustration and are not meant to limit the disclosure or the claims that follow.

### Introduction

Zwitterionic (ZI) polymers are a diverse subclass of materials that are the focus of research for numerous fields including: drug delivery, bio-implants, anti-fouling materials, and electrochemical energy storage.¹⁻⁶ There have been multiple distinct types of zwitterion chemistries and materials that highlight their unique properties and potential for battery electrolytes.⁷⁻¹¹ However, a major disadvantage to widespread use of zwitterions is the limited number of chemistries that are commercially available or easy to synthesize.¹² A variety of chemistries can be found within the literature, but only a handful can be easily purchased commercially, and most are sulfobetaine (SB) zwitterions. Even among those that are available, synthesis can be very difficult and have a low yield. For this reason, there is a need to continue to develop new zwitterion chemistries, particularly containing carboxybetaine (CB) and phosphorylcholine (PC) motifs, which also lower the synthetic barrier and increase zwitterion availability for future applications.

Of the zwitterions that have been studied and synthesized in the literature, a number have been motivated by existing structures found in nature. One of the most well-known commercially available ZI monomers, 2-methacryloyloxyethyl phosphorylcholine (MPC), is inspired by phospholipids found in the membranes of cells.¹³⁻¹⁵ Due to its high bio-compatibility and hydrophilicity, over the years MPC has been used for numerous bio applications like anti-fouling coatings for implants.¹⁶⁻¹⁷ Another recent example of a nature-inspired zwitterion polymer comes from trimethylamine N-oxide (TMAO). An organic osmolyte found in saltwater fishes, TMAO is a new class of zwitterionic material that does not fall into one of the three major categories (carboxybetaine, sulfobetaine, phosphorylcholine). Featuring only a single covalent bond between the cationic and anionic zwitterion moieties, TMAO-derived zwitterionic polymers show extremely high hydrophilicity and antifouling potential that is important for the development of new biomaterials.¹⁸ A naturally occurring zwitterion molecule known as trigonelline (N-methylnicotinic acid) that contains a CB-type carboxylate anion and a pyridinium cation.¹⁹ Found in coffee beans and other plant seeds, trigonelline becomes nicotinic acid when roasted at high temperature and is useful precursor material for the synthesis of new CB-type zwitterionic monomers.²⁰

Two new bio-inspired CB-type zwitterions are synthesized from nicotinic acid with different polymerizable groups. The monomers are then mixed into a lithium-containing ionic liquid electrolyte, and their impact on ion transport performance is compared to several existing chemistries.¹¹ The first monomer, synthesized with allyl bromide, showed a moderate 7Li 1D NMR chemical shift that is only slightly lower than what was observed for CBMA. However, due to the difficulty in polymerizing an allyl group through radical polymerization, an ionogel could not be formed, which resulted in minimal changes to ion transport. In contrast, the second monomer, made with 4-vinylbenzyl chloride (VBC), showed a negligible chemical shift while also exhibiting moderate improvements to lithium conductivity through DC polarization and AC impedance spectroscopy measurements. These results illustrate the importance of a connected polymer network and polyzwitterion solubility on Li⁺ transport in an ionic liquid (IL) environment, and these factors should be considered when designing and synthesizing new zwitterion monomers. Results with the available chemistries demonstrated that zwitterions derived from nicotinic acid are straightforward to synthesize and can have a beneficial impact on the properties of ionogel electrolytes.

### Experimental Methods

### Materials

N-butyl-N-methylpyrrolidinium bis(trifluoromethylsulfonyl)imide (BMP TFSI) (High Purity grade), lithium bis(trifluoromethylsulfonyl)imide (LiTFSI), and 2-hydroxy-2-methylpropiophenone (HOMPP), were purchased from MilliporeSigma and stored in a N₂-filled glove box (H₂O, O₂<1 ppm). Synthesis reagents, nicotinic acid, allyl bromide, 4-vinylbenzyl chloride, anhydrous dimethylformamide (DMF), and tetrahydrofuran (THF) were purchased from Sigma Aldrich. Lithium foil (99.9%, 0.75 mm thick) was purchased from Alfa Aesar and stored in an Ar-filled glove box (H₂O, O₂<0.5 ppm) until preparation of coin cells. Celgard separator (25 μm thickness) and stainless steel (SS) coin cell parts (CR2032) were purchased from MTI Corp.

### Synthesis of CBZI

Nicotinic acid was dissolved in anhydrous DMF at a ratio of 1 to 15 by mol, and stirred at 50° C. until fully dissolved. In low light conditions, allyl bromide is added at a 1:1 molar ratio to nicotinic acid, and the reaction is performed overnight (can be confirmed by a change in color in the solution). Monomer product is recovered by precipitating the DMF solution in THF and cooled in an ice bath until a solid forms. The monomer product is then washed with additional THF, reprecipitated, and dried in a vacuum overnight at low temperature. To make the product into a zwitterion, the monomer is added to a solution of 5 wt % NaOH in H₂O and stirred for at least one hour. The zwitterionic monomer (CBZ1) is finally recovered by precipitation in acetone in an ice bath and drying under vacuum. The final product was dried under reduced pressure at room temperature and stored in a refrigerator until use. NMR spectroscopy was performed in a Bruker AVANCE III 500 MHz NMR spectrometer using D₂O as the solvent. ¹H NMR (D₂O, 500 MHz): 9.14, 8.82, 8.79, 8.01 (m, Pr), 5.9-6.01 (1H, ═CH), 5.35-5.4 (2H, ═CH₂), 5.14 (2H, —CH₂N).

### Synthesis of CBZ2

For synthesis of CBZ2, nicotinic acid is again dissolved in anhydrous DMF at a ratio of 1 to 15 by mol, and stirred at 50° C. until fully dissolved. Due to the reactivity of the monomer, the solution is first cooled to room temperature before 4-vinylbenzyl chloride is (VBC) is added to the solution at a 1:1 molar ratio. The reaction is performed overnight and a visible change in the opacity of the solution (becomes milky white) can be observed. Monomer product is recovered by precipitating the DMF solution in tetrahydrofuran (THF) and cooled in an ice bath until a solid forms. The monomer product is then washed with additional THF, re-precipitated, and dried in a vacuum overnight at low temperature. Precipitation was also attempted in di-ethyl ether, but overall THF proved to be the better nonsolvent that worked well even at room temperature. To make the product into a zwitterion, the monomer is added to a solution of 5 wt % NaOH in H₂O and stirred for at least one hour. The zwitterionic monomer (CBZ2) is recovered by precipitation in acetone at low temperature and dried under vacuum. The final product is stored in a refrigerator until use. NMR spectroscopy of CBZ2 was performed in a Bruker AVANCE III 500 MHz NMR spectrometer using D₂O as the solvent. ¹H NMR (D₂O, 500 MHz): 8.75, 8.41, 8.05, 7.89 (m, Pr), 7.18-7.33 (m, benzene), 6.49-6.55 (2H, ═CH₂), 5.6 (1H, ═CH), 5.16 (2H, —CH₂N).

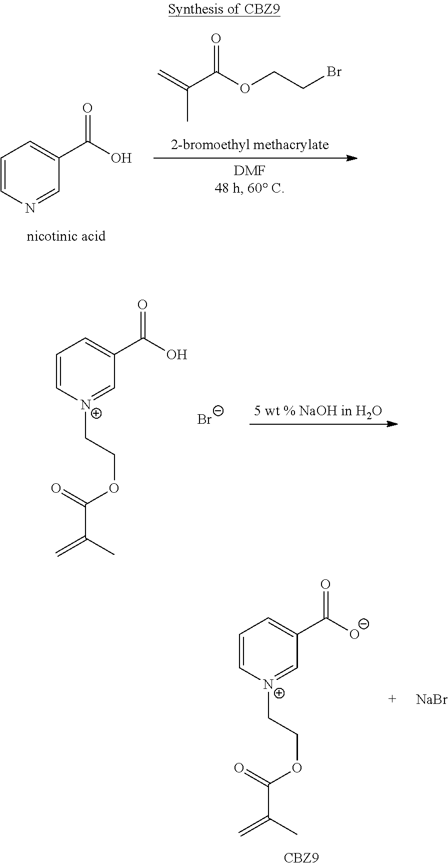

Nicotinic acid was dissolved in anhydrous DMF at a ratio of 1 to 15 by mol, and stirred at 60° C. until fully dissolved. Next, 2-bromoethyl methacrylate was slowly added dropwise to the mixture in a 1.1:1 molar ratio to nicotinic acid and the reaction was carried out for 48 hours (confirmed by a change in color in the solution). Monomer product was recovered by cooling the reaction mixture in an ice bath and precipitating in THF (at a 1:20 ratio) for at least one day. The precipitate was then filtered and dried for one day at room temperature and then dried under vacuum for an additional day. NMR spectroscopy was performed on the acid version of the monomer in a Bruker AVANCE III 500 MHz NMR spectrometer using D₂O as the solvent. ¹H NMR (D₂O, 500 MHz): 9.38, 9.00, 8.93, 8.11, (m, Pr), 5.60-5.96 (2H, ═CH₂), 4.96 (2H, —CH₂O), 4.60 (2H, —CH₂N), 1.73 (3H, —CH₃).

### Preparation of Lithium-Containing Ionic Liquid Electrolytes and Ionogels

A conventional IL/lithium salt solution electrolyte was prepared by dissolving LiTFSI in BMP TFSI at a concentration of 1 M and stirring at 50° C. overnight in a N₂-filled glovebox until a homogeneous solution was obtained. Monomer solutions were prepared by adding a ZI monomer at the desired ZI unit:Li⁺ ratio and stirring overnight. In the case of CBZ2, a clear monomer solution could not be obtained, indicating limited solubility, but polymerization could still proceed. ZI unit:Li⁺ molar ratios ranging from 1:4 to 2:3, corresponding to ZI unit/(ZI unit+Li) mole fractions of 0.2-0.4, were employed in the 1M LiTFSI/BMP TFSI electrolyte (i.e. ZI unit concentrations of 0.25-0.67 M). For clarity, the ZI unit:Li⁺ mole fraction values are used to label the experimental data. To prepare an ionogel, HOMPP photoinitiator (2 wt % monomer basis) was added to a monomer solution, which was stirred for 10 minutes before polymerization was achieved via UV irradiation at 365 nm using a handheld lamp (Spectronic Corp., 8 W) for 10 minutes. Ionogel samples were stored in the glovebox overnight before use.

### Preparation of Coin Cells

Coin cells for DC polarization measurements were prepared by loading a liquid electrolyte into or polymerizing an ionogel within pores of the Celgard separator, in order to standardize the geometry and thickness of the cell electrolyte layer. Electrolyte solutions were infiltrated within Celgard separators (17 mm diameter and 25 μm thickness) under mild vacuum conditions for at least 2 hours (prior to UV irradiation, in the case of ionogel precursor solutions). Lilelectrolyte|Li coin cells were assembled inside an Ar-filled glovebox and discs of Li⁺ metal (˜15 mm diameter) were rolled using a glass vial to brighten the lithium metal surface prior to use. For determination of the temperature dependence of ionic conductivity, SS|electrolyte|SS coin cells were prepared using SS disc electrodes (15.5 mm diameter); electrolytes were confined using an annular Teflon spacer (7.6 mm inner diameter and 1.6 mm thickness) placed between the SS electrodes. All coin cells were sealed using a digital pressure-controlled electric crimper (MTI Corp.).

### Nuclear Magnetic Resonance Spectroscopy Measurements

A Bruker AVANCE III 500 MHz NMR spectrometer with a standard multinuclear broadband observe probe of the z-gradient was used to obtain 1D NMR spectra. Spectroscopy measurements were performed using a relaxation delay of 0.1 ms and a total of 32 scans at room temperature (20° C.), and the nuclei examined were ⁷Li and ¹⁹F to observe the Li⁺ and TFSI⁻ local environments, respectively. A solution of 0.5 M LiTFSI in D₂O was used as reference and locking solution for all samples. All samples were prepared in glass capillary tubes (inner diameter 1.5 mm) that were placed into a standard NMR tube (inner diameter 5 mm) containing the reference solution for the measurements.

### Electrochemical Measurements

All electrochemical measurements were performed using a VersaSTAT 3 potentiostat with a built-in frequency analyzer (Princeton Applied Research). AC impedance spectroscopy was used to measure ionic conductivities of the IL, SIL, and polyzwitterion-supported ionogels of both electrolyte systems. Room temperature ionic conductivity measurements were performed in a N₂-filled glovebox using a custom Teflon cell, and measurements were conducted over a frequency range of 1 Hz to 100 kHz using a sinusoidal voltage amplitude of 10 mV. Temperature-dependent ionic conductivity measurements were performed using symmetric SS|electrolyte|SS coin cells secured to a temperature-controlled microscopy stage (Linkam Scientific Instruments, LTS 420). A holding period of 10 minutes was utilized at each temperature during the heating and cooling cycles to ensure thermal equilibration, and all temperature-dependent Arrhenius model trend lines were fit with a R² value of 0.99 or higher.

The method developed by Bruce and co-workers was used to calculate Li⁺ transference numbers (t_{Li}+) through DC polarization of symmetric Li|electrolyte|Li coin cells. Prior to measurement, cells were preconditioned using a two-hour galvanostatic charge period at 0.01 mA cm⁻², followed by a two-hour potentiostatic hold, and finally a two-hour galvanostatic discharge at −0.01 mA cm⁻². After the preconditioning steps were completed, an additional 12-hour rest period was implemented before any experiments were performed. Determination of Li⁺ transference numbers were conducted via DC polarization/chronoamperometry measurements using an applied potential of 10 mV for two hours, and AC impedance spectra were recorded both before and after the measurements.

### Results and Discussion

### Synthesis and Characterization

In this study, we designed and synthesized two carboxybetaine-based zwitterionic monomers starting from nicotinic acid. In both cases, nicotinic acid was dissolved in anhydrous DMF and reacted with an alkyl halide monomer to create the intermediate cationic product. The monomer was then dissolved in a 5 wt % NaOH aqueous solution to deprotonate the COO⁻ anionic group and make the zwitterion monomers. The synthesis of both monomers and the final chemical structures are outlined in FIG. 1. During synthesis of the monomer in the first step, there is visual confirmation of the product forming by the solution turning an opaque white. Additionally, there is a slight color change visible when making the monomers into zwitterions in the NaOH solution.

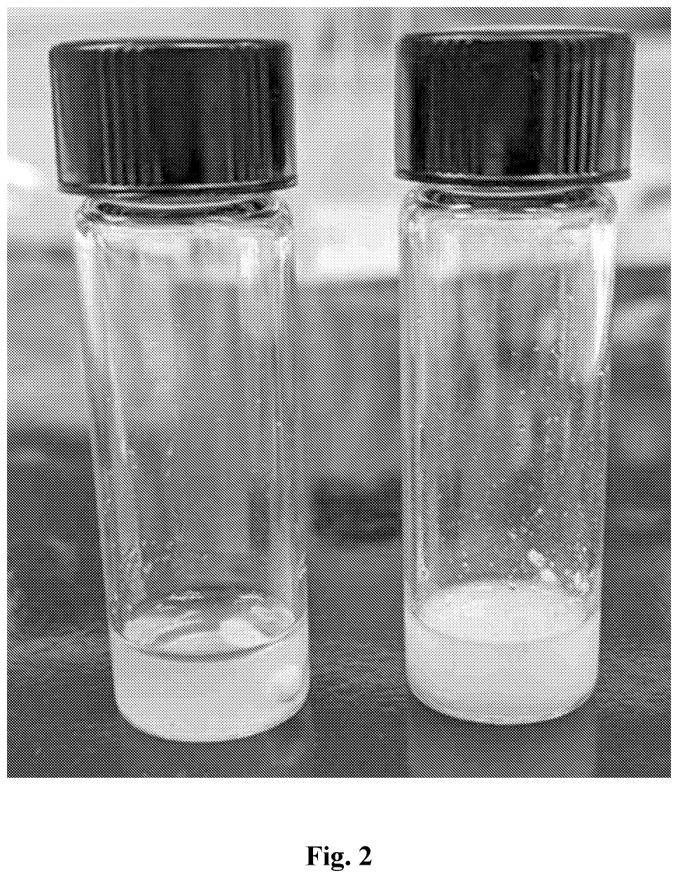

The two monomers synthesized both have the same CB-type zwitterion moieties (pyridinium cation and carboxylate anion), but very different polymerizable groups which affects their solution behavior. In aqueous systems, both monomers are readily soluble (despite the large nonpolar benzyl group on CBZ2) and little difference is observed between the two. However, there are greater differences observed for solubility in a lithium-containing IL electrolyte, and shown in FIG. 2, are solutions of CBZ1 and CBZ2 in 1 M LiTFSI/BMP TFSI. These solutions were picked to have a 0.3 ZI unit:Li⁺ molar ratio and this equates to approximately 3 wt % for CBZ1 and 4.5 wt % for CBZ2. At approximately 3 wt %, CBZ1 shows moderate solubility in 1 M LiTFSI/BMP TFSI and a slight yellow tint that is observable in solution. Lowering the concentration did not lead to a clear solution which suggests some degree of incompatibility between the pyridinium-based cationic group and the specific IL environment. In comparison, the CBZ2 monomer is even less soluble, likely due to the benzene ring, and forms an opaque white solution at all concentrations tested.

Another key difference between these monomers is the nature of the polymerizable groups. The allyl group on CBZ1 is known to be difficult to react through radical polymerization, and all attempts in IL, organic, and aqueous solvent environments using both thermal- and photo-initiators were unsuccessful. As a result, no ionogels could be made with CBZ1 and all subsequent electrochemical measurements in this study were performed with monomer solutions at the specified concentration. In contrast, polymerization was achieved with CBZ2 and this was observed through reduction of the vinyl peaks in ¹H NMR and formation of non-flowing ionogel. Notably, a similar concentration in neat BMP TFSI (without the lithium salt) remained a liquid and did not gel, which may suggest that the Li⁺-ion complexing with the ZI monomer could act as a bridge to form physical crosslinks in the gel.

### ID NMR Chemical Shifts

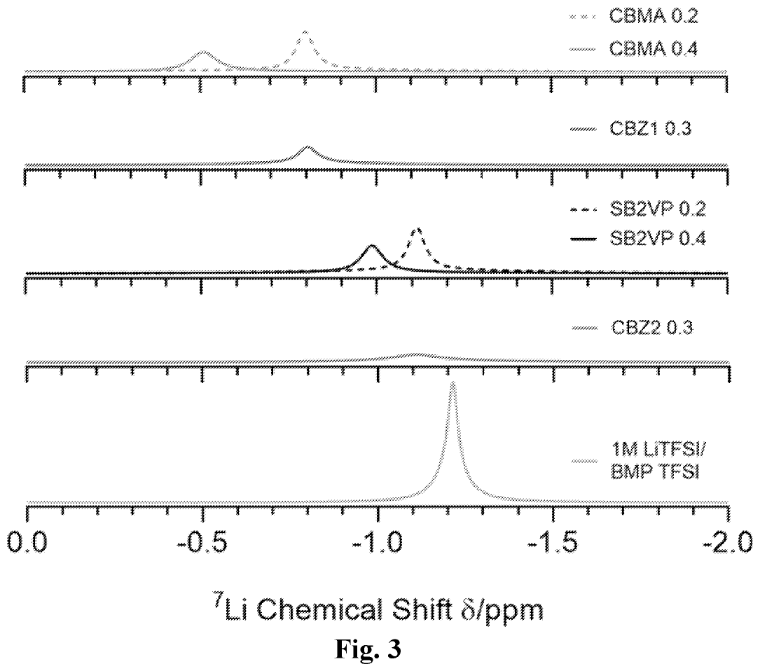

Monomer solutions were screened using 1D NMR chemical shift to probe the interactions between the ZI moieties and Li-ion. By analyzing the ⁷Li chemical shifts of the IL in the presence of different ZI moieties, one can gain insight into changes in the local electron environment. Some other monomer solutions tested were homogeneous solutions that were visibly transparent. By comparison, the nicotinic acid-based zwitterions showed lower solubility in 1 M LiTFSI/BMP TFSI at comparable concentrations. Nevertheless, 1D NMR can still provide useful insights and shown in FIG. 3 are spectra for the IL electrolyte, the two zwitterions synthesized this experiment, and CBMA and SB2VP.

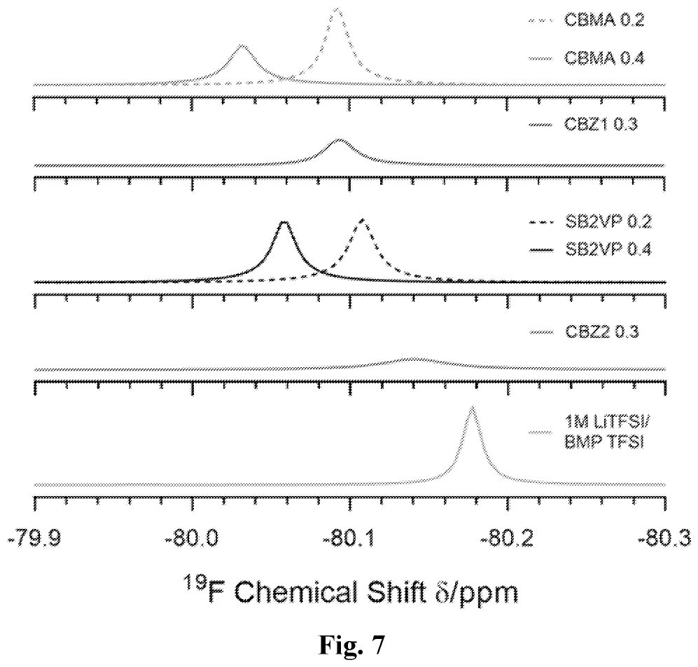

In another study, the largest downfield shifts in the ⁷Li NMR signal peak positions were observed for monomer solutions of CBMA, relative to the peak of the IL electrolyte environment. This shift suggested notable Coulombic interactions between the CB-type zwitterion moiety, and a similar effect is seen with the first newly synthesized ZI monomer, CBZ1. As seen in FIG. 3, CBZ1 yields a moderate downfield ⁷Li peak shift (A6 of ˜0.4 ppm for a ZI unit:Li⁺ mole fraction of 0.3) that is comparable with a slightly lower concentration of CBMA. The difference in chemical shifts may be due to the lower solubility of CBZ1 in the IL or a result of different behaviors of the cationic moieties. However, this result does support our understanding that the anionic COO⁻ group found in CB-type zwitterions can have strong interactions with Li⁺ ions. In comparison, the same is not observed with CBZ2, which not only shows a very small downfield shift that is more comparable to a SB zwitterion (Δδ of ˜0.1 ppm for a ZI unit:Li⁺ mole fraction of 0.3). This difference may arise as a result of the significantly lower solubility of CBZ2 caused by the presence of the benzene ring. Large clumps of undissolved monomer likely limits accessibility of the zwitterion moieties, and reduces the potential for interactions with the Li⁺ ions. A similar trend is observed for the ¹⁹F peak chemical shift (see FIG. 7). This is further supported by the flattening of the peak intensity which can be seen resulting from poor solubility and increased viscosity of the solution. It is speculated that in an IL environment where both ZI monomers are well-dissolved, the chemical shifts would be significantly closer due to identical zwitterion motifs.

### Characterization of Ion Transport

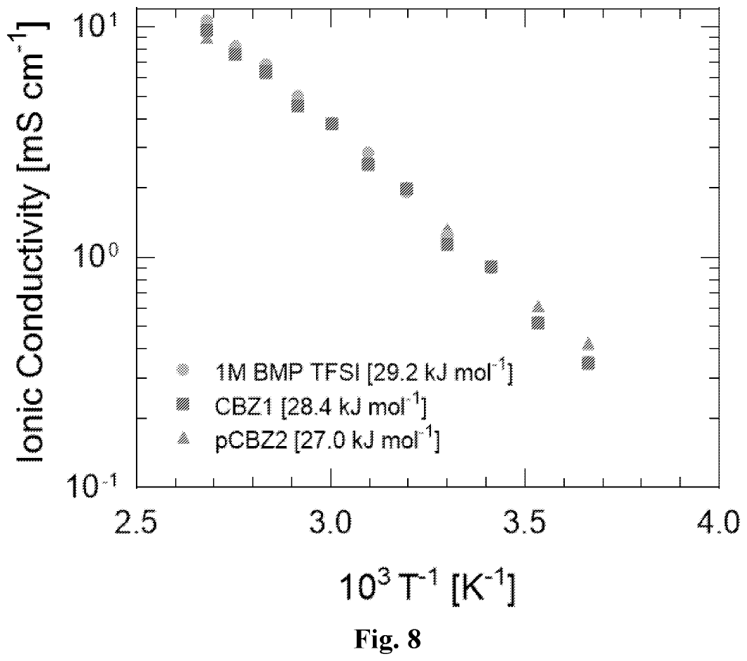

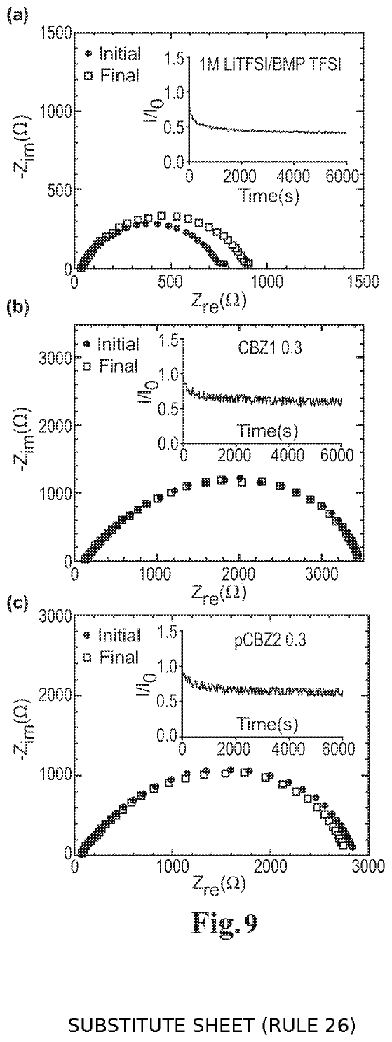

The temperature dependence of total ionic conductivity near ambient conditions (approximately 0° C. to 100° C.) and room temperature lithium-ion transference number values (t_{Li}_{⁺}, the fraction of total current carried by Li⁺ in an applied electric field) were measured for the same electrolyte formulations using AC impedance spectroscopy and DC polarization, respectively. These values are summarized in Table 1 for 1 M LiTFSI/BMP TFSI and the corresponding zwitterion samples. Data for ionogel samples containing pCBMA and pSB2VP are also included for comparison at the same ZI unit:Li⁺ molar ratio. Temperature-dependent ionic conductivity data for new CB-type monomers, as well as DC polarization and AC impedance spectroscopy data used to determine t_{Li}_{⁺} values, were also obtained (see FIG. 8 and FIG. 9). At room temperature, the zwitterion-containing samples exhibited ionic conductivity (a) values are near identical to the neat liquid, suggesting that the ZI groups are promoting a higher degree of ion cluster/pair dissociation in the electrolyte.

More interestingly, despite having comparable total ionic conductivities, there is a notable difference in t_{Li}_{⁺} and Eₐ values for the two new zwitterions. For CBZ1, the values are roughly equivalent to the neat liquid and there is no improvement to lithium transference like what was observed for CBMA. This appears to contradict the previous trend that showed zwitterions with large downfield ⁷Li chemical shifts would also exhibit improved Li-ion transport. However, one potential explanation for why t_{Li}_{⁺} does not change with addition of CBZ1 is because it is not a polyzwitterion sample. Our group hypothesized in a previous study that Li-ion hopping along the polyzwitterion chain is the mechanism for improved Li-ion mobility in a SBVI:MPC copolymer ionogel,⁸ and this may be why the non-polymerizable CBZ1 does not show the same benefit. This is further supported by the t_{Li}_{⁺} and Eₐ values measured for pCBZ2 which show a moderate difference from the neat IL and CBZ1. Despite identical zwitterion moieties, there is a slight decrease in Eₐ and a moderate increase for lithium conductivity observed with pCBZ2, which suggests that the polyzwitterion is crucial for improving ion transport properties. While the lithium conductivity (σ_{Li}_{⁺}) is lower than the highest value achieved using pCBMA, this may be a result of the low solubility of pCBZ2 reducing the effective concentration of available zwitterion. At present, a direct comparison between the two chemistries is difficult, but the improved performance of pCBZ2 compared to the neat IL electrolyte does demonstrate that zwitterions derived from nicotinic acid may be effective for use in ionogel electrolytes.

TABLE 1
Summary of room temperature ionic conductivity (s), activation
energy of total ionic conductivity (Eₐ), lithium-ion transference
number (t_{Li}_{⁺}) and room temperature Li⁺ conductivity
(σ_{Li}_{⁺}) values for the 1M LiTFSI/BMP TFSI electrolyte and
their corresponding polyzwitterion-supported gels.

| | σ [mS cm⁻¹] ± | Eₐ [kJ mol⁻¹] ± | | σ_{Li+} |
|---|---|---|---|---|
| Sample | 1.0 mS cm⁻¹ | 1.0 kJ mol⁻¹ | t_{Li+} | [mS cm⁻¹] |
| 1M LiTFSI/BMP | 0.90 | 29.2 | 0.23 | 0.21 |
| TFSI | | | | |
| pCBMA 0.3 | 1.00 | 24.6 | 0.37 | 0.37 |
| CBZ1 0.3 | 0.91 | 28.4 | 0.26 | 0.24 |
| (monomer) | | | | |
| pCBZ2 0.3 | 0.92 | 27.0 | 0.37 | 0.34 |
| pSB2VP 0.3 | 0.91 | 29.1 | 0.28 | 0.26 |

These CB-type zwitterions, CBZ1 and CBZ2, were tested in 1M LiTFSI/BMP TFSI and compared to some other chemistries. Improvements were observed to lithium conductivity for an pCBZ2-containing ionogel, the low solubility caused by the benzene ring limited interactions between the zwitterion and the IL. This is highlighted by the differences in chemical shifts between CBZ1 and CBZ2 observed through ⁷Li 1D NMR chemical shift—despite having identical zwitterionic motifs, the shift of CBZ1 is closer to CBMA while the CBZ2 shift is minimal. Synthesis is shown in FIG. 4 that uses 2-chloroethyl acrylate as the polymerizable group to make a third nicotinic acid-based zwitterion. Some additional zwitterionic monomer synthesis variations are shown in FIG. 10. Compared to the allyl group found on CBZ1, acrylates are much easier to react through radical polymerization and are chemically similar to the functional group found on some zwitterion monomers. In addition, there is no large non-polar ring present in the structure, so it is expected that solubility will be significantly improved from CBZ2 in 1 M LiTFSI/BMP TFSI. While there may be a lower solubility limit compared to CBMA, it is hypothesized that this acrylate-based zwitterion monomer (CBZ3) will be able to form an ionogel that boosts the Li-ion transport.

Although various embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

### REFERENCES

(1) Blackman, L. D.; Gunatillake, P. A.; Cass, P.; Locock, K. E. S. An Introduction to Zwitterionic Polymer Behavior and Applications in Solution and at Surfaces. Chem. Soc. Rev. 2019, 48, 757-770.
(2) Xuan, F.; Liu, J. Preparation, Characterization and Application of Zwitterionic Polymers Andmembranes: Current Developments and Perspective. Polym. Int. 2009, 58, 1350-1361.
(3) Sun, J.; MacFarlane, D. R.; Byrne, N.; Forsyth, M. Zwitterion Effect in Polyelectrolyte Gels Based on Lithium Methacrylate-N,N-Dimethyl Acrylamide Copolymer. Electrochim. Acta 2006, 51, 4033-4038.
(4) Bengani-Lutz, P.; Converse, E.; Cebe, P.; Asatekin, A. Self-Assembling Zwitterionic Copolymers as Membrane Selective Layers with Excellent Fouling Resistance: Effect of Zwitterion Chemistry. ACS Appl. Mater. Interfaces 2017, 9, 20859-20872.
(5) Leigh, B. L.; Cheng, E.; Xu, L.; Derk, A.; Hansen, M. R.; Guymon, C. A. Antifouling Photograftable Zwitterionic Coatings on PDMS Substrates. Langmuir 2019, 35, 1100-1110.
(6) Lin, X.; Boit, M. O. K.; Wu, K.; Jain, P.; Liu, E. J.; Hsieh, Y. F.; Zhou, Q.; Li, B.; Hung, H. C.; Jiang, S. Zwitterionic Carboxybetaine Polymers Extend the Shelf-Life of Human Platelets. Acta Biomater. 2020, 109, 51-60.
(7) Qin, H.; Panzer, M. J. Zwitterionic Copolymer-Supported Ionogel Electrolytes Featuring a Sodium Salt/Ionic Liquid Solution. Chem. Mater. 2020, 32, 7951-7957.
(8) D'Angelo, A. J.; Panzer, M. J. Decoupling the Ionic Conductivity and Elastic Modulus of Gel Electrolytes: Fully Zwitterionic Copolymer Scaffolds in Lithium Salt/Ionic Liquid Solutions. Adv. Energy Mater. 2018, 8, 1801646.
(9) Taylor, M. E.; Panzer, M. J. Fully-Zwitterionic Polymer-Supported Ionogel Electrolytes Featuring a Hydrophobic Ionic Liquid. J. Phys. Chem. B 2018, 122, 8469-8476.
(10) Taylor, M. E.; Lounder, S. J.; Asatekin, A.; Panzer, M. J. Synthesis and Self-Assembly of Fully Zwitterionic Triblock Copolymers. ACS Mater. Lett. 2020, 2, 261-265.
(11) Taylor, M. E.; Clarkson, D.; Greenbaum, S. G.; Panzer, M. J. Examining the Impact of Polyzwitterion Chemistry on Lithium Ion Transport in Ionogel Electrolytes. 2021, 3, 2635-2645.
(12) Damodaran, V. B.; Murthy, S. N. Bio-Inspired Strategies for Designing Antifouling Biomaterials. Biomater. Res. 2016, 20, 1-11.
(13) Shaplov, A. S.; Marcilla, R.; Mecerreyes, D. Recent Advances in Innovative Polymer Electrolytes Based on Poly(Ionic Liquid)S. Electrochim. Acta 2015, 175, 18-34.
(14) Ueda, T.; Oshida, H.; Kurita, K.; Ishihara, K.; Nakabayashi, N. Preparation of 2-Methacryloyloxyethyl Phosphorylcholine Copolymers with Alkyl Methacrylates and Their Blood Compatibility. Polym. J. 1992, 24, 1259-1269.
(15) Ishihara, K.; Ueda, T.; Nakabayashi, N. Preparation of Phospholipid Polylners and Their Properties as Polymer Hydrogel Membranes. Polym. J. 1990, 22, 355-360.
(16) Feng, W.; Brash, J.; Zhu, S. Atom-Transfer Radical Grafting Polymerization of 2-Methacryloyloxyethyl Phosphorylcholine from Silicon Wafer Surfaces. J. Polym. Sci. Part A Polym. Chem. 2004, 42, 2931-2942.
(17) Ma, Y.; Tang, Y.; Billingham, N. C.; Armes, S. P.; Lewis, A. L. Synthesis of Biocompatible, Stimuli-Responsive, Physical Gels Based on ABA Triblock Copolymers. Biomacromolecules 2003, 4, 864-868.
(18) Li, B.; Jain, P.; Ma, J.; Smith, J. K.; Yuan, Z.; Hung, H. C.; He, Y.; Lin, X.; Wu, K.; Pfaendtner, J.; Jiang, S. Trimethylamine N-Oxide-Derived Zwitterionic Polymers: A New Class of Ultralow Fouling Bioinspired Materials. Sci. Adv. 2019, 5, eaaw9562.
(19) Ashihara, H.; Ludwig, I. A.; Katahira, R.; Yokota, T.; Fujimura, T.; Crozier, A. Trigonelline and Related Nicotinic Acid Metabolites: Occurrence, Biosynthesis, Taxonomic Considerations, and Their Roles in Planta and in Human Health. Phytochem. Rev. 2015, 14, 765-798.
(20) Kalaska, B.; Piotrowski, L.; Leszczynska, A.; Michalowski, B.; Kramkowski, K.; Kaminski, T.; Adamus, J.; Marcinek, A.; Gebicki, J.; Mogielnicki, A.; Buczko, W. Antithrombotic Effects of Pyridinium Compounds Formed from Trigonelline upon Coffee Roasting. J. Agric. Food Chem. 2014, 62, 2853-2860.

### INCORPORATION BY REFERENCE

All U.S. patents, and U.S. and PCT patent application publications mentioned herein are hereby incorporated by reference in their entirety as if each individual patent or patent application publication was specifically and individually indicated to be incorporated by reference. In case of conflict, the present application, including any definitions herein, will control.

### EQUIVALENTS

Those skilled in the art will recognize or be able to ascertain using no more than routine experimentation many equivalents to the specific embodiments of the present invention described herein. Such equivalents are intended to be encompassed by the following claims. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A polymer, comprising a plurality of monomers, wherein at least some of the monomers are zwitterions that comprise a betaine having a pyridinium group and a carboxylate group.

2. The polymer of claim 1, wherein the polymer is a hydrogel.

3. The polymer of claim 1 or 2, wherein said carboxylate group is linked to C3 of said pyridinium group.

4. The polymer of any one of claims 1-3, wherein the zwitterions further comprise an alkyl, allyl, aryl, vinylbenzyl, acrylate, methacrylate, acrylamide, or a methacrylamide group.

5. The polymer of any one of claims 1-4, wherein the zwitterions comprise: 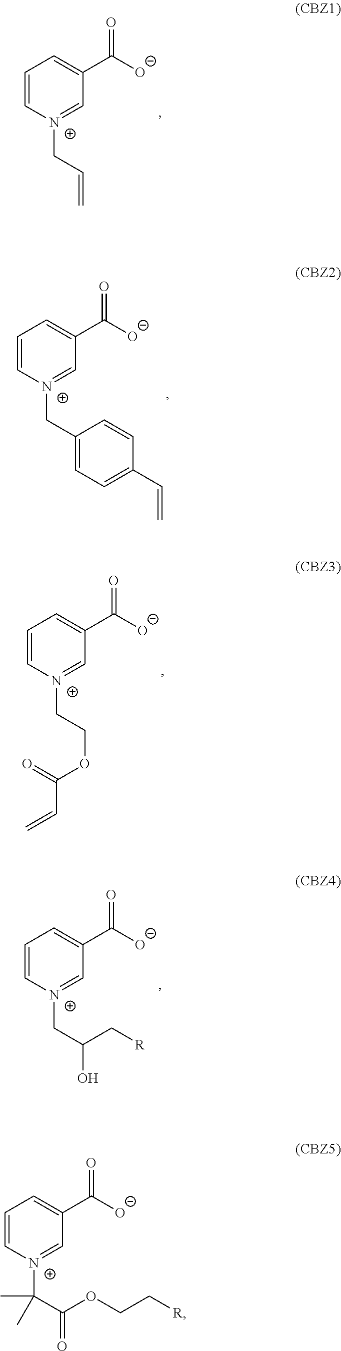 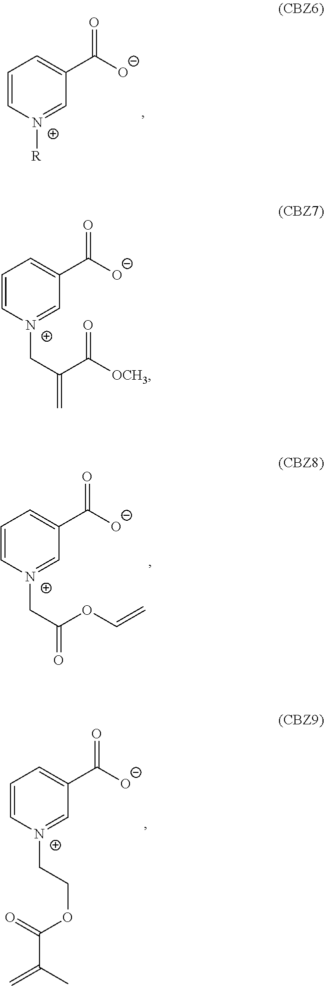
or a combination of any of them; and
R represents acrylate (—OC(O)CH═CH₂), methacrylate (—OC(O)C(CH₃)═CH₂), acrylamide (—NHC(O)CH═CH₂), or methacrylamide (—NHC(O)C(CH₃)═CH₂).

6. The polymer of any one of claims 1-5, wherein the polymer is a copolymer further comprising hydrophobic monomers, charged monomers, ionizable monomers, or a combination of any of them.

7. A filtration membrane, comprising the polymer of any one of claims 1-6.

8. The filtration membrane of claim 7, wherein the membrane is a water filtration membrane.

9. A coating material, comprising the polymer of any one of claims 1-6.

10. The coating material of claim 9, wherein the material is a bio-implant coating material.

11. The coating material of claim 9, wherein the material is an implant surface coating material.

12. The coating material of claim 9, wherein the material is a biomedical device coating material.

13. The coating material of claim 9, wherein the material is an anti-fouling material.

14. A wound-dressing material, comprising the polymer of any one of claims 1-6.

15. An ionic liquid-based electrolyte, comprising the polymer of any one of claims 1-6.

16. The ionic liquid-based electrolyte of claim 15, wherein the electrolyte is an ionogel electrolyte.

17. A polymer electrolyte, comprising the polymer of any one of claims 1-6.

18. A Li-ion battery comprising the electrolyte of any one of claims 15-17.

19. A drug delivery formulation, comprising the polymer of any one of claims 1-6.

20. A method of preparing a carboxybetaine monomer, comprising:
reacting nicotinic acid and an electrophile to obtain a cationic intermediate; and
reacting the cationic intermediate and a base to obtain the carboxybetaine monomer.

21. The method of claim 20, further comprising a solvent.

22. The method of claim 21, wherein the solvent is DMF.

23. The method of any one of claims 20-22, wherein the electrophile is a halide or an epoxide.

24. The method of claim 23, wherein the electrophile is 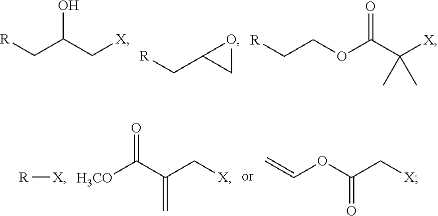
and R is substituted or unsubstituted alkyl, allyl, or vinyl, and X is a halogen.

25. The method of claim 24, wherein the halogen is bromine, chlorine, fluorine, or iodine.

26. The method of claim 23, wherein the electrophile is a halide; and the halide is allyl bromide, 4-vinylbenzyl chloride, or 2-chloroethyl acrylate.

27. The method of any one of claims 20-26, wherein the carboxybetaine monomer is: 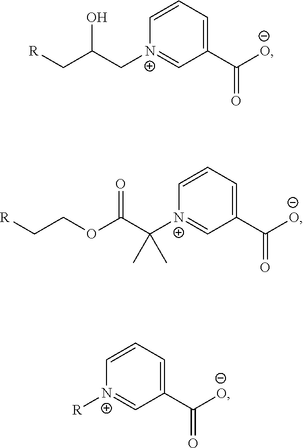 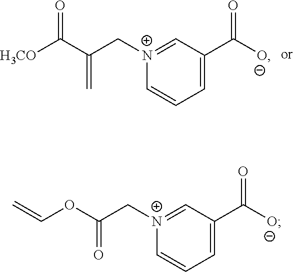
and R is substituted or unsubstituted alkyl, allyl, or vinyl.

28. The method of claim 27, wherein the cationic intermediate is:

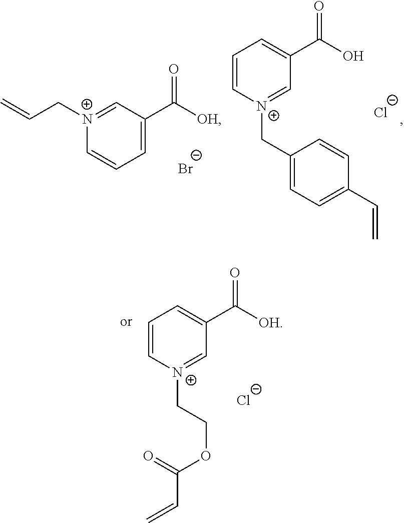

29. The method of any one of claims 20-28, wherein the base is an alkali hydroxide.

30. The method of claim 29, wherein the alkali hydroxide is sodium hydroxide.

31. The method of any one of claims 20-30, wherein the carboxybetaine monomer is:

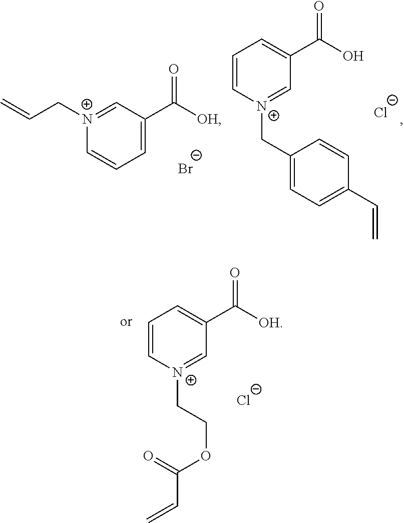

32. A method of preparing a polymer comprising carboxybetaine monomers, comprising:
polymerizing a plurality of carboxybetaine monomers (e.g., carboxybetaine monomers obtained by the method of any one of claims 20-31), thereby preparing the polymer.
